(19) United States
(12) Patent Application Publication
(10) Pub. No.: US 2010/0291710 A1
(43) Pub. Date: Nov. 18, 2010

(54) BIOSENSOR SYSTEM FOR EXTERNAL ACTUATION OF MAGNETIC PARTICLES IN A BIOSENSOR CARTRIDGE

(75) Inventors: Mikhail Mikhaylovich Ovsyanko, Eindhoven (NL); Petrus Johannes Wilhelmus Van Lankvelt, Boekel (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/863,818

(22) PCT Filed: Jan. 22, 2009
(86) PCT No.: PCT/IB2009/050232

§ 371 Date: Jul. 21, 2010

(30) Foreign Application Priority Data
Jan. 28, 2008 (EP) 08100999.5

(51) Int. Cl. G01N 33/543; G01N 35/00

(52) U.S. Cl. 436/518; 422/68.1; 422/82.05

(57) ABSTRACT

The invention provides for a biosensor system (1), wherein the system comprises a biosensor magnet assembly (10) with at least three electromagnetic subunits (20a, 20b, 20c). The magnetic field strength of each electromagnetic subunit is separately changeable by electrical control. Further, the system comprises a detachable biosensor cartridge (30) having a sensor surface and adapted to be arranged with the sensor surface adjacent to the biosensor magnet assembly (10). The invention further provides a method for steering the movement of magnetic particles in a biosensor cartridge (30). The method comprises the steps (a) and (b): in step (a), a sensor surface of a detachable biosensor cartridge (30) is arranged adjacent to a biosensor magnet assembly (10). The biosensor magnet assembly comprises three electromagnetic subunits (20a, 20b, 20c), wherein the magnetic field strength of each subunit is separately changeable by electrical control. In step (b), the magnetic field strength of each subunit is separately changed by electrical control to obtain a magnetic field gradient in the biosensor cartridge (30).

### FIELD OF THE INVENTION

The invention relates to biosensor systems comprising a biosensor cartridge and a biosensor magnet assembly with at least three electromagnetic subunits for use on one side of the biosensor cartridge, and to a method for steering the movement of magnetic particles in the biosensor cartridge.

### BACKGROUND OF THE INVENTION

Various analytical procedures to detect an analyte in a test sample are known. For example, immunoassays use the mechanisms of the immune system, wherein antibodies and the respective antigens are capable of binding to one another. This specific reaction mechanism is used to determine the presence or concentration of the antigen in a test sample.

In particular, the antibody or the antigen (analyte of interest) is labeled to quantify the interactions between antibody and antigen. Common labels are, for example, fluorescent and chemiluminescent molecules, colored particles (beads) or radioisotopes.

Recently, magnetic labels have been used in immunoassays to detect the presence or quantity of an analyte. The use of magnetic labels as, for example magnetic particles (beads), has several advantages. The magnetic particles can be actuated by applying a magnetic field such that the analytical procedure can be accelerated. Further, there is no magnetic background signal in a biological test sample influencing the detection of the magnetic particles.

However, these immunoassays using magnetic labels require means for (a) actuating the magnetic particles bound to the antigens to be immobilized near the sensor surface of the sensor cartridge, and for (b) flushing away the remaining unbound magnetic particles not to influence the quantity measurement of the bound particles. Therefore, for example, two magnets may be arranged on opposite sides of the sensor cartridge, wherein the first magnet attracts the magnetic particles to move through the test sample toward the sensor surface, and then the second magnet attracts unbound magnetic particles to move away from the sensor surface. In this configuration, the two magnets are mounted on a holding structure, and the holding structure mechanically moves the magnets toward or away from the sensor surface (see R. Luxton et al., “Use of External Magnetic Fields to reduce reaction times in an immunoassay . . . ”, Anal. Chem. 2004, 76, 1715-1719).

Such a method is very laborious and time-consuming and needs a complex support system for arranging the two magnets on opposite sides of the sensor cartridge. Further, the first magnet arranged below the sensor cartridge actuates the magnetic particles only in the direction perpendicular to the sensor surface, but not in the horizontal direction (parallel to the sensor surface). Therefore, areas with accumulations of unbound magnetic particles may exist on the sensor surface next to areas with only few or maybe too less magnetic particles to bind with the antigens of interest. This may result in unreliable test results.

### SUMMARY OF THE INVENTION

There is therefore a need to provide a simple biosensor system allowing a time saving operation and an actuation of magnetic particles to steer their movement through the test sample in horizontal direction in order to provide more reliable test results. Further, there is a need to provide a method which allows to accurately control the movement of the magnetic particles in the sensor cartridge.

According to the present invention, at least three electromagnetic subunits are used in a biosensor magnet assembly such that the spatial diffusion of magnetic particles in a biosensor cartridge may be controlled. By adjusting a pre-determined magnetic field gradient in the cartridge, analytes (antigens) included in the test sample and labeled with magnetic particles may be moved toward a sensor surface in the cartridge to bind to immobilized antibodies. The bound complex (sandwich) structure of antibody, antigen (analyte to be tested) and magnetic particle (label) may then be detected at the sensor surface such that the mere presence or even the quantity of the analyte in the test sample may be estimated or determined.

According to the invention, a biosensor system is provided, wherein the system comprises a biosensor magnet assembly with at least three electromagnetic subunits. The magnetic field strength of each electromagnetic subunit is separately changeable by electrical control. Further, the system comprises a detachable biosensor cartridge having a sensor surface and adapted to be arranged with the sensor surface adjacent to the biosensor magnet assembly.

A biosensor cartridge is a container or reservoir for receiving a fluid test sample containing the analyte (antigen) of interest. Usually, the cartridge may have at least one plane base area, particularly a rectangular or circular or elliptical base area . The base area functions as a sensor surface at which the analyte of interest may be analyzed by detection procedures. Preferably, the cartridge or at least the plane base area of the cartridge is made, for example, from cyclo-olefin (co)polmers, polyethylene, polystyrene, polycarbonate, or polymethylmetacrylate to enable an optical analysis of the test sample.

A biosensor cartridge may contain or may receive magnetic or magnetizable particles. “Magnetic” or “magnetizable” particles are influenced by the application of a magnetic field and are magnetically responsive. For example, these particles are attracted or repulsed or have a detectable magnetic susceptibility or induction. In a preferred embodiment, these particles are paramagnetic particles and may be made from metals or metal oxides or composite materials such as ferrites (e.g. magnetite). These particles may be beads or labels and are adapted to bind to the antibody and/or the antigen (analyte of interest). Such a binding can occur directly or via a specific binding member as, for example, a protein sandwiched between the particle and the antibody or antigen. In one embodiment of a biosensor cartridge, antibodies are immobilized via capture reagents at the sensor surface of the cartridge and provide a binding site for the antigen (analyte) labeled with the magnetic or magnetizable particle.

The biosensor magnet assembly of the biosensor system according to the invention comprises at least three electromagnetic subunits. In particular, the electromagnetic subunits may comprise coils having a magnetizable (magnetically responsive) core inside each coil. The core may be made of a ferromagnetic material. The biosensor magnet assembly may be arranged in such a way that one of the poles of each subunit is adjacent to the sensor surface at one of the sides of the biosensor cartridge. In a preferred embodiment, the subunits have a cylindrical shape and the two magnetic poles are present at the two ends of the cylinder (i.e. the base areas of the cylinder). The core of a subunit may have a radius of between 0,5 and 3 mm, preferably 1 mm, and the height of the core may be between 3 and 10 mm, preferably 5 mm.

The magnetic field strength of each subunit is separately changeable by electrical control. “Separately changeable” means that the magnetic field of each subunit can be changed by electrical control independently from any change of the magnetic fields of the other subunits. If the subunits comprise electromagnetic coils as described above, the change of the magnetic field strength of a subunit may be carried out by changing the electric current flowing through the coil of a subunit. In this case, the “electrical control” is meant to be the control of the electric current flowing though the coils.

The biosensor system of the present invention allows for a steering of the movement of magnetic or magnetizable particles (beads, for example labels) in the biosensor cartridge due to the changeable magnetic field of the biosensor magnet assembly. Preferably, the particles can be steered to move directly to the sensor surface of the cartridge to save operation time. Further, an up-concentration of particles at a particular location on the sensor surface may be avoided by separately changing the magnetic field strength of one, two or all of the three electromagnetically subunits: due to a separate control of each of the subunits, the magnetic field gradient (proportional to the force acting on the particles) may be regulated to move the particles in horizontal (parallel to the sensor surface) and/or vertical (perpendicular to the sensor surface) direction.

The area of the sensor surface or the whole volume of the biosensor cartridge may be affectable and/or penetrable by the changeable magnetic field of the subunits. The volume of the cartridge is to be understood as its inner volume (excluding any inlets or outlets for filling-in a test sample) into which a test sample including the analyte can be inserted. The area of the sensor surface of the cartridge is generally the plane base area of the cartridge onto which, for example, the magnetic or magnetizable particles and/or antibodies corresponding to the antigens (analytes) to be determined can be immobilized. Advantageously, the biosensor cartridge is arranged adjacent to the biosensor magnet assembly so that the whole volume of the cartridge is affectable and/or penetrable by the magnetic field of the subunits. In this case, all magnetic or magnetizable particles in the cartridge may be actuated and geometrical constraints within the cartridge are avoided.

Preferably, the subunits are spaced from each other by gaps. The gaps are not necessarily filled with any material but with the ambient air. In a further embodiment, the gaps may be filled with a dielectric material. The dielectric material may be a plastic moulding material, into which the three subunits may be embedded such that the outer shape of each subunit is not apparent. In a preferred embodiment, only one or both of the pole surfaces of each subunit is/are not covered by the dielectric material. The dielectric material may function as an insulator between the subunits and may fix the distance between the subunits such that the subunits are not moveable with respect to each other. Hence, geometrically constraints may further be avoided.

In an embodiment of the invention, the subunits are located on a base structure, preferably on a ferromagnetic yoke. A base structure may enable an easier handling of the biosensor magnet assembly and may further avoid geometrical constraints which may arise if the spaces between the subunits, or the distances between each subunit and the cartridge differ from each other. A ferromagnetic yoke as the base structure may strengthen the magnetic field of the biosensor magnet assembly by concentrating the magnetic field lines of magnetic flux inside in the yoke and thus avoiding losses. In particular, the base structure may have the shape of a cuboid with a length and width of between 3 mm and 10 mm, preferably 5 mm, and a height (i.e. the direction toward the biosensor cartridge) of between 2 and 10 mm, preferably 4 mm.

Preferably, the biosensor system of the invention further comprises control means. The control means may be adapted to separately switch or adjust the magnetic field strength of each subunit by electrical control. In particular, the control means may be switching means to switch the orientation of magnetization in the electromagnetic subunits. In particular, the electromagnetic subunits may comprise coils, preferably having a magnetizable core material in its inside, and the electrical control by which the magnetic field strength of each subunit is changed uses an electric current flowing through the coils. In this case, the switching means is adapted to switch the direction of the current flow in order to switch the orientation of magnetization of the subunit(s). In addition or alternatively, the control means comprise adjusting means adapted to separately adjust each subunit by electrical control to obtain a pre-determined magnetic field gradient in the biosensor cartridge. The magnetic field strength can be increased or decreased separately for each subunit by the adjusting means, for example by increasing or decreasing the electric current in one, two or all of the coils of the subunits. Hence, a pre-determined magnetic field gradient may be obtained in the biosensor cartridge, and the obtained magnetic field gradient may easily be modified by the adjusting means at any time of the analytical procedure.

In particular, the adjusting means is adapted to vary the magnetic field gradient in a direction parallel (horizontal) and/or perpendicular (vertical) to the sensor surface of the biosensor cartridge. Due to the possibility of a separate adjustment of the magnetic field strength for each subunit, the magnetic flux density in the cartridge may be variable. Hence, magnetic or magnetizable particles in the biosensor cartridge may be controllable to move in a particular spatial direction, for example in horizontal direction (parallel to the sensor surface) or in vertical direction (perpendicular to the sensor surface). Thus, the diffusion of the magnetic or magnetizable particles in the cartridge may be controlled and accelerated or even slowed-down in each spatial direction by using the adjusting means for changing the magnetic field strength of one, two or all of the three electromagnetic subunits.

In a particular embodiment, the biosensor system of the invention is a FTIR (Frustrated Total Internal Reflection, cf. the description of FIG. 3) magnetic biosensor system. Due to the fact that optical beams and magnetic fields do generally not interfere with each other, optical detection methods for analyzing the presence and preferably the quantity of an analyte of interest in a test sample are advantageous if using magnetic or magnetizable particles as labels. Hence, external magnetic actuation may be well-suited for use with optical detection methods, since sensor disturbances by the magnetic field may be avoided.

The invention further provides a method for steering the movement of magnetic particles in a biosensor cartridge. The method comprises the steps (a) and (b): in step (a), a sensor surface of a detachable biosensor cartridge is arranged adjacent to a biosensor magnet assembly. The biosensor magnet assembly comprises three electromagnetic subunits, wherein the magnetic field strength of each subunit is separately changeable by electrical control. In step (b), the magnetic field strength of each subunit is separately changed by electrical control to obtain a particular magnetic field gradient distribution in the biosensor cartridge. The method may allow to control the movement of magnetic or magnetizable particles in a biosensor cartridge to accelerate the analytical procedure. Further, the analytical procedure may be more reliable, since an equal distribution of the particles in horizontal and/or vertical direction of the cartridge may be achieved by changing the magnetic field strength of one, two or all of the at least three magnetic subunits.

In a particular embodiment, the biosensor magnet assembly and the biosensor cartridge have the same features as described above in connection with the biosensor system of the invention. In a further particular embodiment, the biosensor magnet assembly and the biosensor cartridge used in the method of the invention are parts of the biosensor system as described above.

Preferably, the magnetic field gradient obtained in the biosensor cartridge is variable in a direction parallel and/or perpendicular to the sensor surface of the cartridge. Besides an equal distribution of particles in the cartridge, also a pre-determined non-uniform distribution may be achievable by varying the magnetic field gradient distribution. For example, the particles may be actuated by the gradient to arrange in continuous or discontinuous lines or areas on the sensor surface in the biosensor cartridge to calibrate the detection procedure of one or more particle lines (areas).

In an embodiment of the invention, the method further comprises the following steps: a fluid sample including magnetizable or magnetic particles is provided into the biosensor cartridge, and the magnetic field strength of at least one of the subunits is separately changed by electric control to modify the obtained magnetic field gradient distribution in the biosensor cartridge. A further changing of the magnetic field strength of at least one subunit may allow a re-adjusting and refining of the magnetic field gradient distribution to adapt the gradient to particular circumstances in the biosensor cartridge. For example, some magnetic or magnetizable particles tend to conglomerate such that—first of all—the particles have to be dispersed (for example by providing a strong gradient in horizontal direction of the cartridge, parallel to the sensor surface), and then the separated particles can be steered to move toward the sensor surface in the cartridge, e.g. by providing a strong gradient in vertical direction of the cartridge, i.e. perpendicular to the sensor surface.

In a particular embodiment of the invention, the biosensor cartridge is detached such that the sensor surface of the biosensor cartridge is no longer arranged adjacent to the biosensor magnet assembly. By detaching the biosensor cartridge, for example after an optical measurement of the presence or quantity of the analyte (antigen) of interest is carried out, the biosensor cartridge may be placed in a detection or analyzing means to further test the sensor surface in the cartridge or even to restudy the measurements, for example to calibrate the results. “Detaching” means that the cartridge is not broken or damaged due to the removing of the cartridge from the biosensor system. The cartridge may also be disposed and replaced with another new cartridge.

These and other aspects of the invention will be apparent from and exemplified with reference to the embodiments described hereafter.

### BRIEF DESCRIPTION OF THE DRAWINGS

FIG. 1 schematically shows a set-up for a biosensor system according to one embodiment of the invention;

FIG. 2 shows the calculation of the gradient of B² for the configuration in FIG. 1;

FIG. 3 schematically shows the set-up for a FTIR biosensor system according to one embodiment of the invention.

### DETAILED DESCRIPTION OF EMBODIMENTS

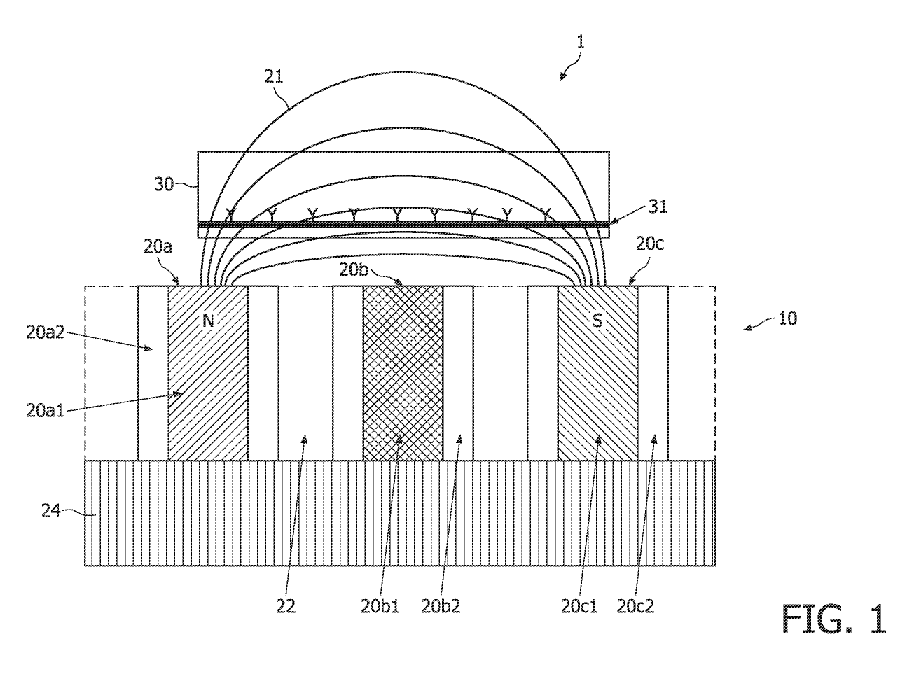

FIG. 1 shows an embodiment of a biosensor system 1 comprising a biosensor magnet assembly 10 with e.g. three electromagnetic subunits 20a, 20b, and 20c which may be arranged on a base structure 24. In a preferred embodiment, each subunit comprises a coil 20a2, 20b2, 20c2 and a core 20a1, 20b1, 20c1 inside the coil. By changing the electric current flowing through the coil, the magnetic field strength of each subunit may be electrically controlled. In a particular embodiment, the subunits are spaced from each other by gaps 22. Generally, thee three subunits 20a to 20c are arranged on one side of the cartridge 30 facing the three subunits. In case a base structure 24 is used, the detachable cartridge 30 is located adjacent to the subunits such that the biosensor magnet assembly 10 is located between the base structure 24 and the cartridge 30. The gaps 22 may be filled with a dielectric material, e.g. plastics material. Further, the cartridge 30 is arranged such that its volume is affectable and/or penetrable by the magnetic field of the biosensor magnet assembly. In FIG. 1, the electromagnetic subunit 20a has North orientation magnetization N, the subunit 20c has South orientation magnetization S, and the subunit 20b is preferably neutral. Some of the corresponding magnetic field lines 21 of magnetic flux between the first and the third subunit are schematically shown in FIG. 1; the lines of (merely minor) magnetic flux between the neutral subunit 20b and the first and second subunits 20a, 20b are not shown for clarity reasons. The magnetic field strength of each electromagnetic subunit 20a, 20b, or 20c is separately adjustable by electrical control.

According to an embodiment of the method of the present invention, the sensor surface 31 of the cartridge 30 is arranged adjacent to the biosensor magnet assembly 10 in FIG. 1, such that the magnetic field resulting from the three electromagnetic subunits 20a, 20b, and 20c penetrates the whole volume of the cartridge 30. After that, the magnetic field strength of each subunit may be adjusted by electrical control to change a magnetic field gradient within the biosensor cartridge 30. By varying the magnetic field strength of one, two or all of the three electromagnetic subunits, the gradient in the cartridge varies in a particular direction, for example, parallel and/or perpendicular to the sensor surface 31 of the cartridge 30. Magnetic or magnetizable particles present in the cartridge 30 or filled in the cartridge after the gradient is adjusted diffuse through the cartridge according to the force actuating them, for example, to move toward the sensor surface. While the particles move to the sensor surface of the cartridge or before detecting the presence of the particles at the sensor surface, the magnetic field gradient in the cartridge 30 may again be adjusted, for example to further refine the gradient and thus the controlled diffusion of the particles. After analyzing the sensor surface 31 of the cartridge by a detector (not shown in FIG. 1), the cartridge 30 may be detached from the biosensor system 1 for disposal; alternatively, further measurements may be carried out by placing the detached cartridge 30 into a separate detection system.

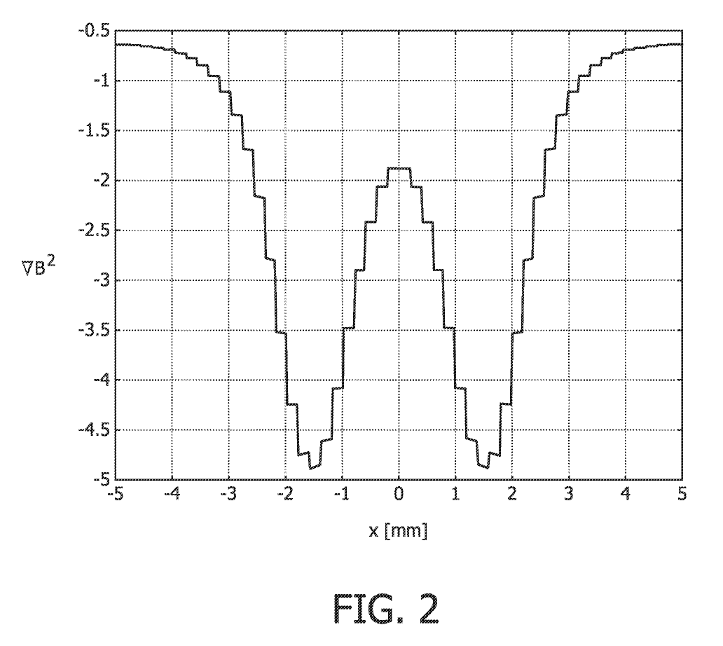

FIG. 2 shows magnetic field gradient calculations for the same configuration as shown in FIG. 1: the left subunit 20a has North orientation magnetization N, the subunit 20c has South orientation magnetization S, and the subunit 20b is neutral. The center of the neutral subunit 20b present between the first and third subunit 20a and 20c is placed at x=0 [mm] in the x-y coordinate system shown in FIG. 2. The calculations are performed at a distance of 1 mm above the poles of the three subunits. Further, a negative value for the magnetic field gradient on the y-axis means that the force (proportional to ∇B²) acting on the magnetic particles, acts downwards in the cartridge, toward the sensor surface. It becomes apparent from FIG. 2 that the absolute value of the gradient is small for x corresponding to the x-coordinate of a subunit, whereas the absolute value of the magnetic field gradient (proportional to the force) for x-coordinate values between those of the subunits is high. Hence, the movement of the particles may be controllable by changing the magnetic field strength of the electromagnetic subunits, i.e. by changing the currents of the coils 20a2, 20b2, and 20c2.

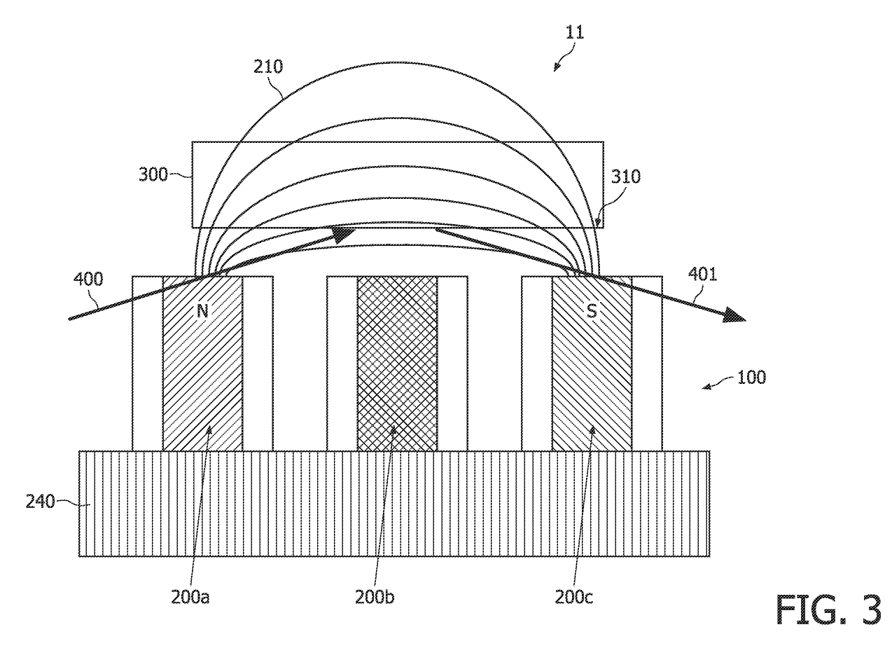

FIG. 3 shows a FTIR (Frustrated Total Internal Reflection) biosensor system 11 comprising a biosensor magnet assembly 100 with three electromagnetic subunits 200a, 200b, and 200c, each preferably comprising a coil and a core inside the coil to electrically control the magnetic field strength of the subunit. The subunits are located on a base structure 240. The detachable cartridge 300 is located adjacent to the subunits so that the biosensor magnet assembly 100 is placed between the base structure 240 and the cartridge 300. Similar to the set-up as shown in FIG. 1, the electromagnetic subunit 200a has North magnetization orientation N, the subunit 200c has South magnetization orientation S, and the subunit 200b is neutral. Some of the magnetic field lines 210 of magnetic flux between the first and the third subunit are schematically shown in FIG. 3. Additionally to the configuration in FIG. 1, FIG. 3 schematically shows a light beam 400 emitted from a light source (not shown), for example a laser or a LED, coupled into the sample at an angle of total internal reflection at the surface 310 of the cartridge facing the biosensor magnet assembly 100. If there are no particles close to the sensor surface 310 of the biosensor cartridge 300, the light is completely reflected. However, if magnetic particles are present close to the sensor surface 310 within the cartridge, the condition to fulfill total internal reflection is violated. As a consequence thereof, a portion of the light is scattered into the cartridge, and the amount of light reflected by the sensor surface 310 is thus decreased. By measuring the intensity of the reflected light 401 with an optical detector (not shown in FIG. 3), it is possible to estimate the amount of magnetic particles being close to the sensor surface 310 of the cartridge 300.

While the invention has been illustrated and described in detail in the drawings and foregoing description, such illustration and description are to be considered illustrative or exemplary and non-restrictive; the invention is thus not limited to the disclosed embodiments. Variations to the disclosed embodiments can be understood and effected by those skilled in the art and practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures can not be used to advantage. Any reference signs in the claims should not be considered as limiting the scope.

1. A biosensor system (1; 11) comprising:
(a) a biosensor magnet assembly (10; 100) comprising
three electromagnetic subunits (20a, 20b, 20c; 200a, 200b, 200c),
wherein the magnetic field strength of each electromagnetic
subunit is separately changeable by electrical control; and
(b) a detachable biosensor cartridge (30; 300) comprising a sensor surface (31;
310) adapted to be arranged adjacent to the biosensor magnet assembly
(10; 100).

2. The biosensor system of claim 1, wherein the whole volume of the biosensor cartridge (30; 300) is affectable by the changeable magnetic field generated by the subunits.

3. The biosensor system of claim 1, wherein the subunits (20a, 20b, 20c; 200a, 200b, 200c) are spaced from each other by gaps (22).

4. The biosensor system of claim 3, wherein the gaps (22) are filled with a dielectric material.

5. The biosensor system of claim 1, wherein the subunits are located on a common base structure (24; 240).

6. The biosensor system of claim 5, wherein the base structure (24; 240) is a ferromagnetic yoke.

7. The biosensor system of claim 1, further comprising
(c) control means adapted to separately switch or adjust the magnetic
field strength of each subunit.

8. The biosensor system of claim 7, wherein the control means is adapted to generate a pre-determined magnetic field gradient in the biosensor cartridge (30; 300).

9. The biosensor system of claim 8, wherein the control means is adapted to vary the magnetic field gradient in a direction parallel and/or perpendicular to the sensor surface (31; 310) of the biosensor cartridge (30; 300).

10. The system of claim 1, wherein the system (1; 11) is a FTIR magnetic biosensor system.

11. A method for steering the movement of magnetic particles in a biosensor cartridge (30; 300), comprising the steps of
(a) arranging a sensor surface (31; 310) of a detachable biosensor cartridge
adjacent to a biosensor magnet assembly (10; 100), the biosensor magnet
assembly comprising
three electromagnetic subunits (20a, 20b, 20c; 200a, 200b, 200c),
wherein the magnetic field strength of each subunit is separately
changeable by electrical control,
(b) changing the magnetic field strength of each subunit separately by
electrical control to obtain a magnetic field gradient in the biosensor
cartridge (30; 300).

12. The method of claim 11, wherein the magnetic field gradient is variable in a direction parallel and/or perpendicular to the sensor surface (31; 310) of the biosensor cartridge (30; 300).

13. The method of claim 11, further comprising:
providing a fluid sample including magnetizable or magnetic particles into
the biosensor cartridge (30; 300);
changing the magnetic field strength of each subunit separately by electric
control to modify the obtained magnetic field gradient in the biosensor
cartridge.

14. The method of claim 11, further comprising:
detaching the biosensor cartridge (30; 300).
